(19) United States
(12) Patent Application Publication
(10) Pub. No.: US 2024/0404700 A1
(43) Pub. Date: Dec. 5, 2024

(54) SYSTEM AND METHOD FOR PERSONALIZED TREATMENT PRIORITIZATION

(71) Applicant: The Royal Institution for the Advancement of Learning/McGill University, Montreal (CA)

(72) Inventors: John TRAKADIS, Mount Royal (CA); Lin Qi, Montreal (CA); Sameer Sardaar, Montreal (CA)

(73) Assignee: The Royal Institution for the Advancement of Learning/McGill University, Montreal (CA)

(21) Appl. No.: 18/679,017

(22) Filed: May 30, 2024

Related U.S. Application Data
(60) Provisional application No. 63/505,210, filed on May 31, 2023.

(51) Int. Cl. G16H 50/20; G16H 20/00

(52) U.S. Cl. CPC G16H 50/20; G16H 20/00

(57) ABSTRACT

Methods and systems for treatment prioritization using a neural network model are provided. Training the model includes generating an enriched genetic knowledge graph with edges characterizing relationships between genetic variants, genes, diseases, treatments and symptoms, embedding the graph vertices in genetic knowledge vectors, receiving training patient feature value sets, generating a training dataset of training patient vectors labelled with target values comprising treatment efficacity by scaling the genetic knowledge vectors by the patient feature value sets, and training the model to process an input patient vector and generate a predicted efficacity of the at least one treatment option. Performing inferences includes receiving patient feature values characterizing a patient, generating the patient input vector by scaling the genetic knowledge vectors by the patient feature values, and providing the patient input vector as input to the model, thereby generating a treatment efficacy vector predicting efficacity of a plurality of treatment options.

### CROSS-REFERENCE TO RELATED PATENT APPLICATION

This application claims the benefit of, and priority to, U.S. Provisional Patent Application No. 63/505,210, filed 31 May 2023, and entitled “System and Method for Personalized Treatment Prioritization”, the disclosure of which is hereby incorporated by reference in its entirety.

### TECHNICAL FIELD

The technical field relates to precision medicine, and more specifically to systems and methods for prioritizing treatment options based on patient characteristics.

### BACKGROUND

Current diagnostic criteria and treatment options do not adequately factor in genetic variations at the individual patient level. This results in major costs and burdens at the patient and societal level. Ineffective treatment and side effects of non-optimal treatment are associated with annual costs of around $495-672 billion in the US (i.e., 16% of total US health care expenditures in 2016). For example, 10-30% of patients with schizophrenia show no response to conventional antipsychotics, with 30-60% of patients experiencing side effects and little improvement. The identification of effective treatments, within a class of medications, for patients relies mostly on trial and error. Often, several medications, targeting a given symptom/disease, need to be tried before the right medication is found, causing excessive burden on the patients. Precision medicine (PM) seeks to individualize the treatment of patients. It aims to enable patient-centric treatment and prevention of disease based on integration of data on individual variations in genetics, lifestyle, and environment. Advances in PM could be promising in identifying effective treatments and reducing the need for trial and error.

Pharmacogenomics combines pharmacology with genomics with the aim of understanding how genes affect responses to drugs. Advances in this field have enabled predictions of drug efficacy and side effects in individuals with highly complex and heterogenous genetic disorders, such as psychiatric diseases. Genetic associations of medication usage have been identified via genome-wide association studies. Wu, Y., et al., Genome-wide association study of medication-use and associated disease in the UK Biobank. Nat Commun, 2019. 10 (1): p. 1891, identified 505 linkage disequilibrium independent genetic loci significantly associated with self-reported medication use from 23 medication categories utilizing self-reported medication-use data from the UK biobank (UKBB). Moreover, numerous pharmacogenetic (PGx) variants and genes are associated with medication response based on the literature, underlining the important role of genetic variations in medication response and usage patterns.

The importance of genetic variants to human diseases has been well established beyond pharmacogenomics studies. Thousands of genomic loci have been implicated through genome-wide association studies (GWAS) and have advanced our understanding of the genetic basis of many diseases. Moreover, there are different genetic prediction tools which are based on polygenic risk scores (PRS). PRS aim to predict a person's risk of developing a particular disease or condition based on their genetic profile. These scores are calculated using a person's genetic data and a statistical model that considers the known GWAS associations of a phenotype. The result is a numerical score that indicates the person's relative risk of developing the phenotype, with higher scores indicating a higher risk and lower scores indicating a lower risk. However, PRS have explained only a small fraction of the genetic heritability of many diseases, and the applicability of GWAS and PRS results to individual patients has been limited.

There are some important limitations to PRS and other current approaches in the context of precision medicine. First, the impact of each variant on a given patient is considered independently of the genomic background of that patient, i.e., without accounting for heterogeneity of effects, and combined, potentially non-linear, effects of multiple variants (e.g., epistasis, gene interactions). Moreover, PRS approaches are often hypothesis-free, meaning that the analysis results of genetic variants are purely statistical, without the contribution of prior biological knowledge.

### SUMMARY

In accordance with an aspect, a method for treatment prioritization is provided. The method includes generating an enriched genetic knowledge graph representing relationships between genetic variants, genes, diseases, symptoms and/or treatments, the enriched genetic knowledge graph including vertices and edges, with each vertex corresponding to one of a genetic variant, a gene, a disease, a treatment option and a symptom, analyzing the enriched genetic knowledge graph to embed the vertices of the enriched genetic knowledge graph in corresponding genetic knowledge vectors, each genetic knowledge vector encoding information about the corresponding vertex, the corresponding vertex's surrounding graph structure and/or the corresponding vertex's relationship with other vertices, receiving a plurality of training patient feature value sets characterizing a plurality of patients, generating a plurality of training patient vectors, each training patient vector being generated by scaling the genetic knowledge vectors by one of the plurality of training patient feature value sets, generating a training dataset including at least a subset of the plurality of training patient vectors, each training patient vector being labelled with a target value including an efficacity of at least one treatment option, and training a treatment prioritization neural network model using the training dataset to process an input patient vector and generate a predicted efficacity of the at least one treatment option.

In accordance with another aspect, a method for treatment prioritization is provided. The method includes receiving patient feature values characterizing a patient, generating the patient input vector by scaling the genetic knowledge vectors by the patient feature values, and providing the patient input vector as an input to the treatment prioritization neural network model, thereby generating a treatment efficacy vector predicting efficacity of a plurality of treatment options.

In accordance with a further aspect, a system for treatment prioritization is provided. The system includes one or more processor, memory having a treatment prioritization neural network model stored thereon, and a training module. The training module is configured to cause the one or more processor to generate an enriched genetic knowledge graph representing relationships between genetic variants, genes, diseases, symptoms and/or treatments, the enriched genetic knowledge graph including vertices and edges, with each vertex corresponding to one of a genetic variant, a gene, a disease, a treatment option and a symptom, analyze the enriched genetic knowledge graph to embed the vertices of the enriched genetic knowledge graph in corresponding genetic knowledge vectors, each genetic knowledge vector encoding information about the corresponding vertex, the corresponding vertex's surrounding graph structure and/or the corresponding vertex's relationship with other vertices, receive a plurality of training patient feature value sets characterizing a plurality of patients, generate a plurality of training patient vectors, each training patient vector being generated by scaling the genetic knowledge vectors by one of the plurality of training patient feature value sets, generate a training dataset including at least a subset of the plurality of training patient vectors, each training patient vector being labelled with a target value including an efficacity of at least one treatment option, and train the treatment prioritization neural network model using the training dataset to process an input patient vector and generate a predicted efficacity of the at least one treatment option.

In accordance with yet another aspect, a system for treatment prioritization is provided. The system includes one or more processor, memory having a trained treatment prioritization neural network model stored thereon, an input device configured to receive from a user patient feature values characterizing a patient, and an inference module. The inference module is configured to cause the one or more processor to generate a patient input vector by scaling genetic knowledge vectors by the patient feature values, and provide the patient input vector as an input to the treatment prioritization neural network model, thereby generating a treatment efficacy vector predicting efficacity of a plurality of treatment options. The system further includes an output device configured to convey to the user the predicted efficacity of the plurality of treatment options.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment.

FIGS. 1A and 1B are flowcharts illustrating a method for training a treatment prioritization neural network and for obtaining a personalized treatment prioritization for a patient using said neural network, according to an embodiment.

FIG. 2 shows AUC performance metrics of an embodiment as described below to predict treatment efficacy and of two possible alternative embodiments.

FIGS. 3A to 3J show the accuracy of the treatment ranking according to an embodiment described below for 10 common medications.

FIGS. 4A and 4Y show a uniform manifold approximation and projection of patients affected by 25 common diseases according to feature impact values, in accordance with an embodiment described below.

FIGS. 5A and 5Y show a uniform manifold approximation and projection of patients affected by the same 25 common diseases as FIGS. 4A to 4Y according to raw feature values.

FIG. 6A is a schematic illustrating a system for training a treatment prioritization neural network, according to an embodiment.

FIG. 6B is a schematic illustrating a system for obtaining a personalized treatment prioritization for a patient using a treatment prioritization neural network, according to an embodiment.

### DETAILED DESCRIPTION

It will be appreciated that, for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practised without these specific details. In other instances, well-known methods, procedures and components have not been described in detail so as not to obscure the embodiments described herein. Furthermore, this description is not to be considered as limiting the scope of the embodiments described herein in any way but rather as merely describing the implementation of the various embodiments described herein.

One or more systems described herein may be implemented in computer program(s) executed on processing device(s), each comprising at least one processor, a data storage system (including volatile and/or non-volatile memory and/or storage elements), and optionally at least one input and/or output device. “Processing devices” encompass computers, servers and/or specialized electronic devices which receive, process and/or transmit data. As an example, “processing devices” can include processing means, such as microcontrollers, microprocessors, and/or CPUs, or be implemented on FPGAs. For example, and without limitation, a processing device may be a programmable logic unit, a mainframe computer, a server, a personal computer, a cloud-based program or system, a laptop, a personal data assistant, a cellular telephone, a smartphone, a wearable device, a tablet, a video game console or a portable video game device.

Each program is preferably implemented in a high-level programming and/or scripting language, for instance an imperative e.g., procedural or object-oriented, or a declarative e.g., functional or logic, language, to communicate with a computer system. However, a program can be implemented in assembly or machine language if desired. In any case, the language may be a compiled or an interpreted language. Each such computer program is preferably stored on a storage media or a device readable by a general or special purpose programmable computer for configuring and operating the computer when the storage media or device is read by the computer to perform the procedures described herein. In some embodiments, the system may be embedded within an operating system running on the programmable computer.

Furthermore, the system, processes and methods of the described embodiments are capable of being distributed in a computer program product comprising a computer readable medium that bears computer-usable instructions for one or more processors. The computer-usable instructions may also be in various forms including compiled and non-compiled code.

The processor(s) are used in combination with storage medium, also referred to as “memory” or “storage means”. Storage medium can store instructions, algorithms, rules and/or trading data to be processed. Storage medium encompasses volatile or non-volatile/persistent memory, such as registers, cache, RAM, flash memory, ROM, diskettes, compact disks, tapes, chips, as examples only. The type of memory is, of course, chosen according to the desired use, whether it should retain instructions, or temporarily store, retain or update data. Steps of the proposed method are implemented as software instructions and algorithms, stored in computer memory and executed by processors.

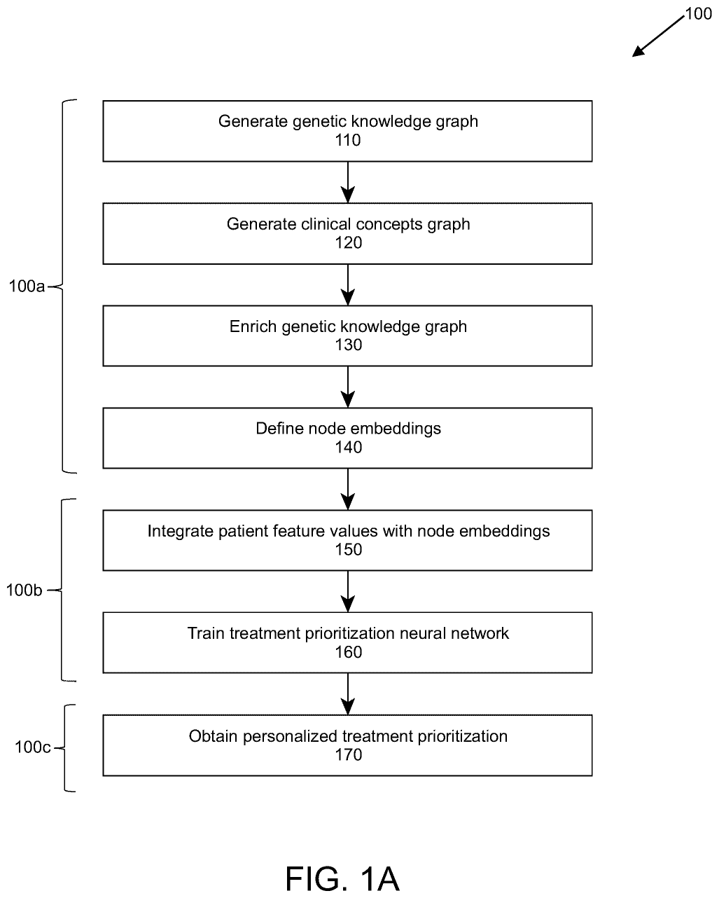

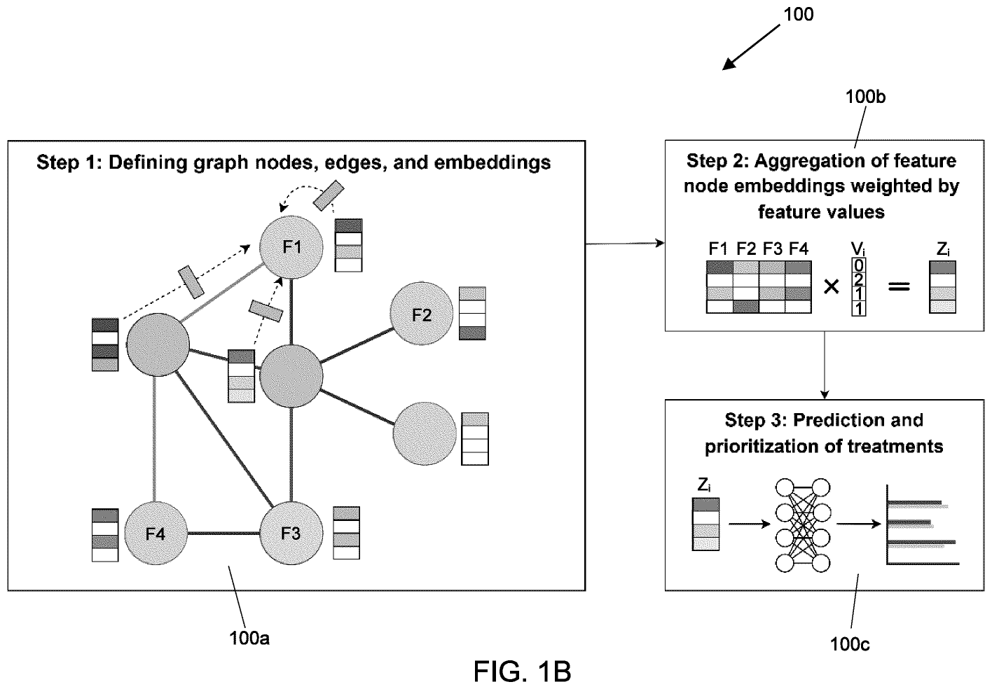

With reference to FIGS. 1A and 1B, an exemplary method 100 for training a treatment prioritization neural network and for obtaining a personalized treatment prioritization for a patient using said neural network is shown. Broadly described, the method 100 can be divided into three main steps. A first step 100a can involve defining graph nodes (or vertices), edges, and embeddings. A simplified example of a graph with nodes, node embeddings, and edges between nodes is shown in FIG. 1B. This step creates the genetic knowledge graph foundation for subsequent graph representation learning. A second step 100b can involve aggregating feature node embeddings and corresponding feature values for a patient into a single aggregated vector. In FIG. 1B, an example is shown with nodes F1-F4 (i.e., feature nodes), and the corresponding feature values (Vi) for a patient to yield the aggregated vector Zi. Finally, a third step 100c can involve feeding the aggregated vector Zi into a prediction model (e.g., neural network) to obtain predictions on desired target values about each treatment. As can be appreciated, each step 100a, 100b, 100c can include substeps as described below.

In substep 110, a genetic knowledge graph can be created. As an example, the genetic knowledge graph can be a heterogeneous graph, i.e., a graph consisting of multiple types of nodes and edges. Nodes in the graph can be connected to other nodes based on specific domain knowledge. The genetic knowledge graph can provide a graph representation of multiple types of biomedical entities based on known relationships from prior biomedical knowledge. This knowledge can serve as a source of prior information for machine learning models in the task of treatment prediction/prioritization.

The genetic knowledge graph can comprise nodes (i.e. vertices) and edges, with each node corresponding to one of a genetic variant, a gene, a disease and a treatment option. In its basic form, the genetic knowledge graph can include some of the following node types: genetic variants, genes, diseases and treatments. In some embodiments, genetic variant nodes are labelled with a genetic variant name or identifier such as a Variant Call Format, gene nodes are labelled with a gene name or identifier such as a HUGO Gene Nomenclature Committee ID, disease nodes are labelled with a disease name or identifier such as a International Classification of Diseases code, and treatment nodes are labelled with a treatment name or identifier such as a Anatomical Therapeutic Chemical code. In some embodiments, node labels can correspond to strings and/or concept/class identifiers associated for instance with a classification, a hierarchy or an ontology. The graph can also include edge types between all possible combinations of node types. Edges between nodes can be created on existing knowledge of their relationships. For instance, an edge can connect a treatment known to be used for a given disease; an edge can connect a genetic variant and gene if the genetic variant occurs in or around the gene; edges between genes can exist if the gene products are known to physically interact with one another. As another example, an edge or an arc can connect two nodes on the same type to represent a known semantic relationship, for instance extracted from a classification, hierarchy or ontology. It can be appreciated that different other semantics can be associated with different types of edges. In some embodiments, edges are labelled or coloured with semantic information. These relationships and others can for instance be obtained from publicly available databases (e.g., PharmGKB, DisGeNET), manually curated, and/or automatically extracted from scientific literature (e.g., PubMed), for instance by using natural language processing, knowledge extraction, information extraction, data extraction and/or information retrieval.

In some embodiments, disease subtype nodes can be included in the genetic knowledge graph. In some embodiments, the process of adding disease subtypes nodes can be automated, for instance as per the following method. First, a disease prediction machine learning model predicts the likelihood of patients developing a particular disease. A disease prediction model can be trained for this purpose, for instance a regression model, or an off-the-shelf model can be used. The contribution of each feature to a prediction (i.e., feature impact values) are then calculated, for instance using the integrated gradients technique. Computing the feature contribution can include computing the gradient of the output with respect to each feature and integrating the gradients over the path from a predefined baseline value to the actual value of each feature. Patient stratification is performed using the feature impact values as the input to a clustering algorithm in order to extract subgroups of patients with similar feature impact values. The resulting clusters obtained from patient stratification represent more homogenous groups of patients with a given disease, which can be deemed to correspond to disease subtypes, allowing for improved treatment prediction/prioritization. Finally, disease subtype nodes are created and edges (or arcs) between disease nodes and disease subtype nodes, or between disease subtype nodes, are created to incorporate the information as part of the genetic knowledge graph.

In substep 120, clinical patient information can be integrated into the genetic knowledge graph to create an enriched genetic knowledge graph. The clinical patient information includes for instance medical records of a number of patients treated at a health facility. In some embodiments, the clinical patient information can be anonymized. Much of clinical information is present as textual clinical patient information, e.g., unstructured text data. Accordingly, the textual data can be processed to extract biomedical and clinical concepts therefrom. Information from unstructured text data (clinical notes and/or text data from patients themselves) can be integrated into the genetic knowledge graph described above. After extracting biomedical and clinical concepts from the unstructured text data, a network representation of the concepts can be built. As an example, the following steps can be used. Named-entity recognition systems (i.e., named-entity recognizers) can be used to identify mentions of diseases, symptoms, and treatments (i.e., extracted clinical concepts) from clinical texts. The recognized entities, i.e., the extracted clinical concepts, can then be converted into a graph, referred to herein as the clinical concepts graph, for instance by representing each identified concept as a node and representing relationships between at least diseases, symptoms and treatments identified from the clinical patient information as edges between disease nodes, symptom nodes and treatment nodes. Edges between nodes can be created to represent relationships, for instance based on the cooccurrence of recognized clinical concept entities within the same clinical texts. For example, based on a clinical note that mentions a patient's symptoms and the diseases they have been diagnosed with, nodes can be created for the symptoms, and these nodes can be connected with an edge to the disease nodes to represent the relationship between them.

Once the clinical concepts graph has been created, the next step can involve integrating the clinical concepts graph with the genetic knowledge graph. The genetic knowledge graph can consist of the following node types: genetic variants, genes, diseases, and treatments. The clinical concepts graph can for instance include the following node types: diseases, symptoms, and treatments. The genetic knowledge graph and the clinical concepts graph can therefore share at least common disease and common treatment nodes, e.g., if a disease is associated with one node in each of the two graphs. Integrating the clinical concepts graph with the genetic knowledge graph can involve merging vertices of the genetic knowledge graph and vertices of the clinical concepts graph that are common, e.g., that correspond to one of a common disease and a common treatment option. As an example, two nodes can be deemed to be common if they share the same type and the same label. In some embodiments, nodes can be deemed to be common if they have similar labels. As an example, similarity can be defined based on a string similarity metric and/or based on a semantic distance, e.g., if the labels correspond to identifiers associated with a classification, a hierarchy, an ontology, etc. In some embodiments, integrating two graphs G₁= [symbol] V₁, E₁ [symbol] and G₂= [symbol] V₂, E₂ [symbol] to generate a new, e.g., enriched graph G₃= [symbol] V₃, E₃ [symbol] includes performing a union of the two graphs G₁∪G₂. In some embodiments, the union is a non-disjoint union, resulting in G₃= [symbol] V₃, E₃ [symbol] =G₁∪G₂= [symbol] V₁∪V₂, E₁∪E₂ [symbol] . In some embodiments, integration of the two graphs can be performed by: first creating a mapping between treatment nodes shared between the genetic knowledge graph and clinical concepts graph; then creating a mapping between disease nodes shared between the genetic knowledge graph and clinical concepts graph; and finally creating new edges (representing relationships) between diseases and genes, and diseases and variants based on information publicly available from variant-disease and gene-disease association databases.

This above procedure will combine the information from the clinical concepts graph with the information from the genetic knowledge graph yielding an enriched genetic knowledge graph with a more complete and comprehensive view of health knowledge from both a genetic and clinical perspective, and allows the treatment prediction method to leverage novel patterns/insights that would not be possible with the individual graphs alone.

A further substep 130 can include integrating other biological and biomedical data in the form of text (e.g., journal articles) or graphs (e.g., biochemical pathways) into the genetic knowledge graph, for instance by using the same approach as described above for the clinical concepts graph and genetic knowledge graph.

A substep 140 can include embedding one, some or all nodes of one of the graphs defined above in a structure more suited to computation, for instance in a vector. It can be appreciated that a vector embedding encoding information from a graph can be stored using less memory and manipulated by a computer faster and using fewer computational resources than the graph it encodes. In some embodiments, graph neural networks (GNN), i.e., a neural network trained to use a graph as input, such as graph convolutional networks (GCN), can be used to analyze the network representation of the genetic knowledge graph capturing both genetic and clinical data to define a vector embedding. This involves learning an embedding (vector) for each node in the network. The node embedding encodes information about a given node, a given node's surrounding graph structure, and its relationships with other nodes. The process of learning the node embedding for each node is illustrated at 100a in FIG. 1B for node F1.

The node embedding can be initialized based on any prior attributes of a node, such as properties describing the node entity. In some embodiments, the properties of genetic variants, including the chromosome, position, surrounding sequence, expected consequence, and protein alteration, encoded as a vector are included as the initial node embeddings of genetic variant nodes. In some embodiments, a text description of any node, encoded as a vector using any suitable method, for instance bag-of-word, term frequency-inverse document frequency, Word2vec and/or a neural network-based language model, e.g., a transformer model like Bidirectional Encoder Representations from Transformers, is included as the initial node embeddings. In some embodiment, where no prior attributes or descriptions of nodes are available, the node embeddings are initialized with learnable random parameters which are updated (“optimized”) during the training process.

The embedding (vector) for a given node X can be learned through a series of graph convolutions as part of training the GCN. Graph convolutions are normalized aggregations of the transformed embeddings of the neighbouring nodes of the given node X, with its own transformed node embedding. The transformed node embeddings in the graph convolution process are obtained through a transformation by a learnable neural network. In some embodiments, these transformations are specific to each edge type in the network (i.e., a different neural network for each edge type). In some other embodiments, these transformations use the same (neural network) for all edge types.

Multiple layers of transformations and aggregations can be performed recursively, with each layer capturing increasingly complex patterns and relationships in the surrounding graph structure of each node. This allows the GCN to learn a distributed representation for each node that encodes rich information of the node's position and relationships in the graph.

Once the genetic knowledge graph has been generated and enriched and the embeddings for each node for the GCN have been defined, it is possible, in a subsequent substep 150, to integrate the unique patient feature values of a patient or of a plurality of patients with embeddings (vectors) of nodes corresponding to the features (i.e., feature node embeddings) to create a patient vector. Patient vectors created at this substep can be used both as part of a training set for training a treatment prioritization neural network, and as the input to the neural network for obtaining a personalized treatment prioritization for a patient. The general process involves a scaling (multiplication) of the feature node embeddings by the feature values and a subsequent aggregation into a single vector.

As an example, a number N of nodes can be defined as the feature nodes. Each feature node can be embedded in a vertical vector of size M, and all the created vectors can be combined, e.g., by concatenation in an M×N matrix. Patient feature values can be represented for each patient in a vertical vector of size M. The feature node matrix embedding can then be scaled by the patient feature values and aggregated to yield each aggregated vector, which can be named “patient vector”.

A simplified example of the process is shown at 100b in FIG. 1B. Nodes F1-F4 are defined as the feature nodes. In the box for Step 2, The feature node embeddings are scaled by the patient feature values (Vi) and aggregated to yield the aggregated vector Zi.

As examples only, the following feature nodes and feature value categories can be defined.

Genetic variant nodes: in some embodiments, the corresponding feature value can correspond to the number of copies of a variant, e.g., values 0, 1 or 2, as derived from genotyping or genome sequencing data; in some embodiments, the original genotype values can be normalized or transformed via a neural network into transformed genotype values, and the transformed genotype value can then be used as the feature value for a given genetic variant node.

Clinical concepts nodes: in some embodiments, the corresponding feature value can be 0 or 1, to indicate the presence or absence of the clinical concept; in some embodiments, the feature value can be a continuous number over a suitable range, e.g., from −1 to 1, or a normalized value, indicating the presence of the clinical concept and the associated degree of positive or negative sentiment of the clinical concept. The sentiment associated with each mention of a clinical concept in a clinical text can for instance be extracted with a natural language processing system, an information extraction system or an information retrieval system, e.g., a sentiment analysis system. For example, a mention of the drug metformin could be extracted from a clinical note, associated with the sentiment of “poor response”, which could then be translated to a negative numerical value.

Disease nodes (and, in some embodiments, disease subtype nodes): in some embodiments, disease nodes are grouped under the category of clinical concept nodes, with feature values defined the same way; in some embodiments, the corresponding feature value is the likelihood of the disease as derived from external prediction models or tools.

Overall, this step yields a feature value-weighted aggregated vector, named “patient vector”, which is a fixed-length vector summary of the individual's features and the subset of feature nodes induced by those features. In some embodiments, the aggregation is a sum operation over all the feature value-weighted node embeddings. In some embodiments, the aggregation is a concatenation operation of the feature value-weighted node embeddings, followed by a transformation with a neural network.

At substep 160, the neural network parameters and the node embeddings defined in the GCN can be optimized during a supervised training process.

A training dataset can be constructed with multiple patients and their patient feature values, for instance corresponding to the feature categories defined above. Each training sample can then be labelled by defining the target values of each patient in the training dataset for the treatment prediction task. In some embodiments, the target values can include a vector with values of 0 or 1, where each position in the vector corresponding to a specific treatment, a value of 1 indicating that a patient has found the corresponding treatment to be effective. In some embodiments, the target values can additionally or alternatively include a vector with values of 0 or 1, where each position in the vector corresponds to a specific treatment, a value of 1 indicating that a patient has found the corresponding treatment to have side effects.

The aggregated embedding training patient vectors defined above can then be provided as input into a classifier, e.g., a neural network trained to output target predictions for each patient. A differentiable loss function (e.g., binary cross-entropy) can then be defined to measure the difference between the target predictions and the target values. This loss function can be used to evaluate the performance of the prediction model, and to guide the optimization of the neural network parameters and node embeddings. Since all of the operations in the previous steps are differentiable, the parameters of all the neural networks and embeddings defined in the model can be jointly optimized using iterations of gradient descent and backpropagation. This allows the prediction model to learn from the training data and improve its performance over time, leading to more accurate predictions of the target values.

It is understood that the neural networks can be implemented using computer hardware elements, computer software elements or a combination thereof. Accordingly, the neural networks and additional submodules described herein can be referred to as being computer-implemented. Various computationally intensive tasks of the neural network can be carried out on one or more processors (central processing units and/or graphical processing units) of one or more programmable computers. For example, and without limitation, the programmable computer may be a programmable logic unit, a mainframe computer, server, personal computer, cloud-based program or system, laptop, personal data assistant, cellular telephone, smartphone, wearable device, tablet device, virtual reality device, smart display devices such as a smart TV, set-top box, video game console, or portable video game device, among others.

The neural network trained in substep 160 makes it possible to output target predictions corresponding to whether a patient finds a treatment option to be effective and/or whether the patient experiences side effects with a treatment option. This can be used at substep 170 to provide a ranking system taking as input the raw target predictions over multiple treatments and prioritizing the most relevant treatments for a target patient.

For example, a patient vector can be generated which characterizes the target patient. The patient vector can be generated by scaling the genetic knowledge vectors by corresponding patient feature values, and aggregating the scaled genetic knowledge vectors, as described above when generating vectors for the training dataset. The patient vector can be provided as an input to a treatment prioritization neural network trained according to the substeps described above, thereby generating a treatment efficacy vector predicting efficacity of a plurality of treatment options and, in some embodiments, a side effect vector. In some embodiments, the output of the classifier is a real vector including a predicted efficacy value for a number of treatment options each associated with one cell of the vector or, in some embodiments, a predicted risk value of the patient experimenting side effects for a number of treatment options. The efficacy value and/or side effect risk value can for instance be a real number between 0 and 1. In some embodiments, the efficacy value and/or side effect risk value can correspond to a predicted provability of a treatment option being effective and/or causing side effects for a patient. In some embodiments, the sum of the vector elements is 1. For each treatment, the raw output prediction for a given patient can be subtracted from the mean of the raw output predictions over all patients (from the training dataset) and divided by the standard deviation of the raw output probabilities over all patients to yield a standardized Z-score, e.g., with a mean of 0 and a standard deviation of 1. This standardization can help to make the predictions for the different treatments comparable. The standardized Z-scores of all treatments can then be sorted to obtain a final ranking of the treatments for the patient. This personalized ranking aids the treating physician to select the treatment of a given patient. For example, upon being presented the rank treatments, the physician can proceed with applying the treatments according to their rank.

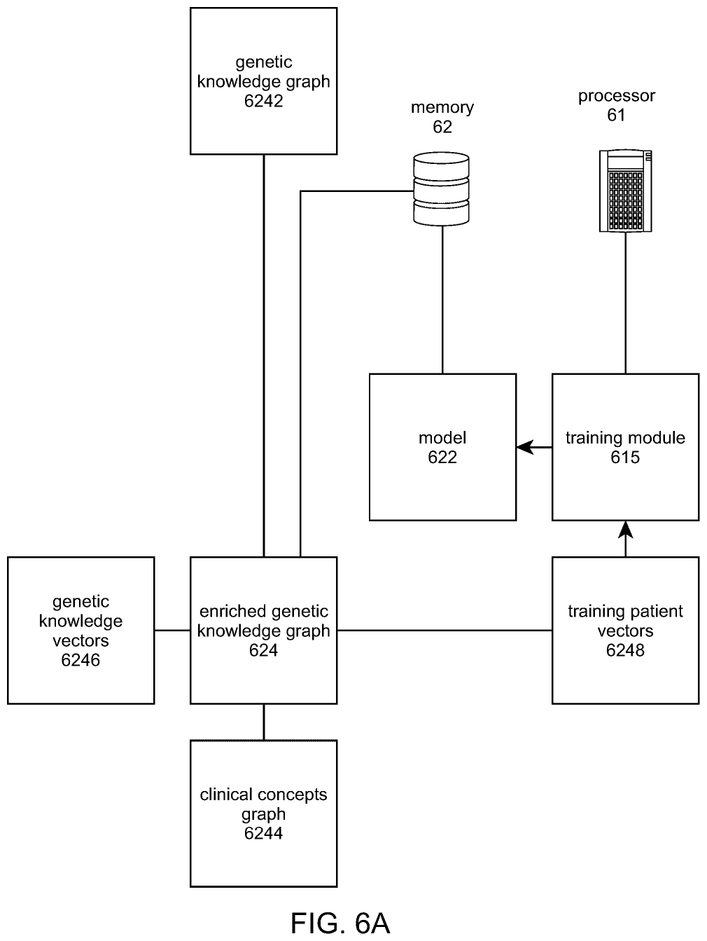

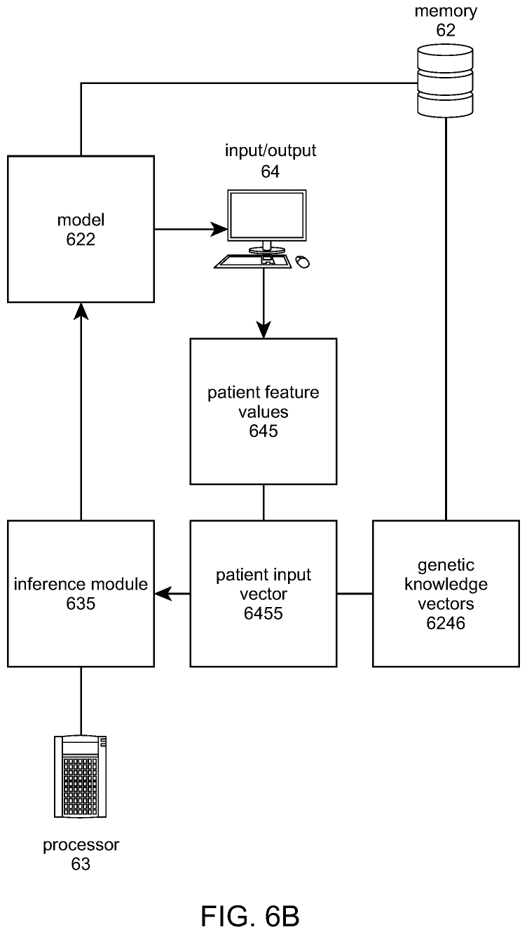

With reference to FIG. 6A, an exemplary system for training a treatment prioritization neural network is shown. Broadly described, the system includes a processor 61 executing instructions that implement a training module 615 and a memory 62 storing the treatment prioritization neural network model 622.

The system includes a training module 615 configured to cause the processor 61 to carry out steps for training a treatment prioritization neural network as described above. For example, the training module 615 can first cause a genetic knowledge graph 6242 and a clinical concepts graph 6244 to be integrated into an enriched genetic knowledge graph 624, as described above. The training module 615 causes a neural network to embed nodes of the enriched genetic knowledge graph 624 into corresponding genetic knowledge vectors 6246, as described above. The training module 615 causes the genetic knowledge vectors 6246 to be scaled by training patient feature value sets to obtain training patient vectors 6248, as described above. The training patient vectors 6248 can then be used to train the model 622, as described above. In some embodiments, the system includes a named-entity recognizer to obtain knowledge and/or patient information from text, as described above.

With reference to FIG. 6B, an exemplary system for obtaining a personalized treatment prioritization for a patient using a trained treatment prioritization neural network is shown. Broadly described, the system includes a memory 62 storing the treatment prioritization neural network model 622 and the genetic knowledge vectors 6246, a processor 63 executing instructions that implement an inference module 635, and one or more input and/or output devices 64 to obtain patient feature values 645 and/or convey predications to a user.

The system includes an inference module 635 configured to cause the processor 63 to carry out steps for obtaining a personalized treatment prioritization for a patient as described above. For example, a user of the system can enter patient feature values 645 related to a patient for whom they wish to obtain a prediction, for instance at an input device 64. The inference module 635 causes the genetic knowledge vectors 6246 to be scaled by the patient feature values 645, resulting in a patient input vector 6455, as described above. The patient input vector 6455 is suitable for input in the treatment prioritization neural network model 622. The inference module causes the patient input vector 6455 to be processed by the treatment prioritization neural network model 622 to obtain predictions, as described above. The predictions can then be conveyed to the user, for instance at an output device 64. In some embodiments, the inference module 635 causes the predictions to be normalized and/or ranked, for instance by computing a Z-score of the output values with respect to training values and by ranking the Z-scores, as described above. The predictions can be conveyed to the user according to their rank via the output device 64.

It can be appreciated that the systems of FIGS. 6A and 6B can be equivalently implemented as one, or a plurality of systems, and that processors 61 and 63 can be the same processor or can be different processors. Moreover, it can be appreciated that each of processors 61 and 63 can correspond to a plurality of processors implementing the training module 615 and/or the inference module 635, for instance for parallel or distributed processing.

An embodiment of the method and systems as described above was tested using the UK biobank (UKBB), which includes circa 500,000 individuals.

To prioritize treatment, pharmacogenomic information can be used to identify which medications are most likely to be effective for a given patient, based on their genetic profile. This allows doctors to select treatments that are tailored to the patient's specific genetic makeup, increasing the likelihood of success and minimizing adverse effects.

In the first step of this process, a heterogenous graph of biomedical node types was generated. This is done using open-sourced biomedical domain knowledge from PharmGKB. PharmGKB is an openly available pharmacogenomics database providing information about the role of genetic variants in drug response. The database also includes genes, drugs, and drug-gene interactions. Data from PharmGKB was processed to including genetic variants, genes, medications, haplotypes, and diseases as part of the genetic knowledge graph. The graph also includes edge types between the node types, representing the relationships between the different node types.

For each node in the graph, a node embedding was initialized with random parameters. The transformations of the graph convolution process were performed using a single neural network for all edge types. Two layers of graph convolutions were defined to capture the surrounding graph structure of each node.

From the graph, a specific set of 3,890 variants and 156 haplotypes were selected as features. The corresponding feature values were extracted from a dataset of 485,754 individuals. No clinical concept features were included since clinical notes were not available for the set of patients. The dataset was split into training, validation, and testing sets, with 70% of the data used for training, 10% for validation, and 20% for testing.

The target values used for optimizing the neural network and embedding parameters are the medications that the individuals in the dataset were regularly taking, out of a total of 264 medications. The target values were encoded as a 264-dimensional vector, with a value of 1 indicating that an individual is taking the medication, and 0 otherwise.

A binary cross-entropy loss function was defined, and the parameters of all neural networks and embeddings were optimized jointly using iterations of gradient descent and backpropagation on the training data. This allowed the system to learn patterns and relationships in the data, and to make predictions about the likelihood of different medications being effective for a given patient based on their genetic profile.

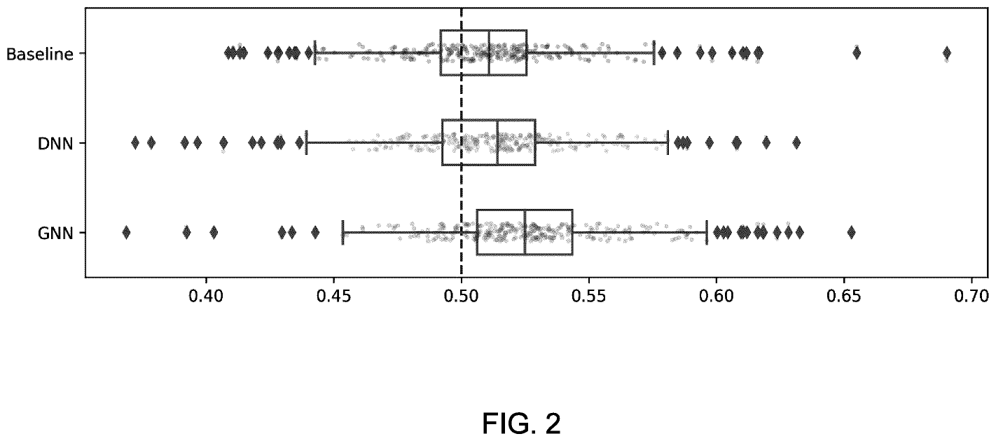

To assess the GCN model performance, it was compared with a baseline logistic regression (baseline) and a dense neural network (DNN). The evaluation metric used to assess and compare model performance is the area under the receiver operating characteristic curve (AUC) on the testing set. FIG. 2 shows an overall AUC performance comparison for all 264 medications for the baseline, DNN and GCN approaches (labelled GNN in FIG. 2). The graph approach performed better overall compared to the DNN, and the baseline models.

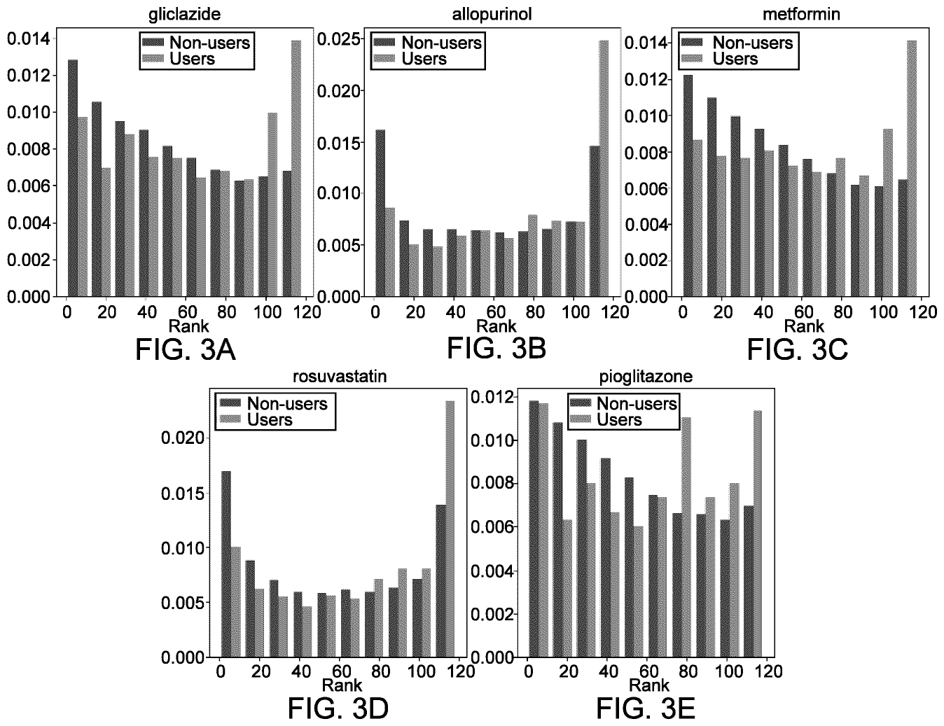

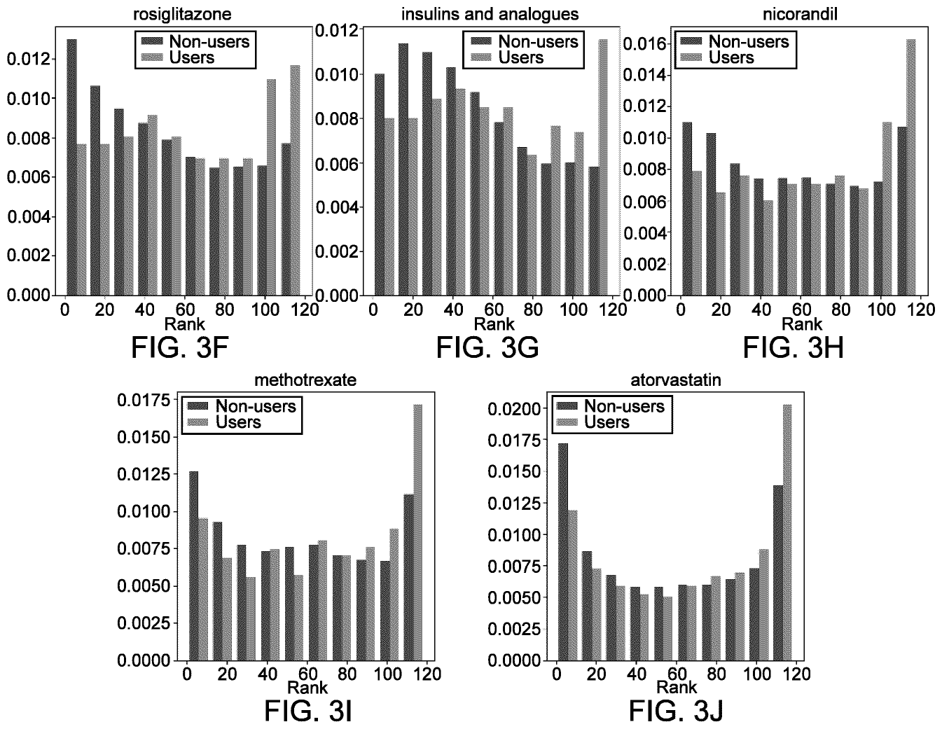

The predicted likelihood of each medication for a patient was transformed into a ranking among all predicted medication efficacy likelihoods. An example demonstrating the effectiveness of the ranking approach is shown for ten medications in FIGS. 3A to 3J. The top ten medications as ranked by AUC values are shown. It can be appreciated from each of FIGS. 3A to 3J that the users of a medication ranked higher on average for those medications than. non-users. This suggests that the proposed method is capable of identifying patterns which are effective for prioritizing medications most likely to be needed by the patient.

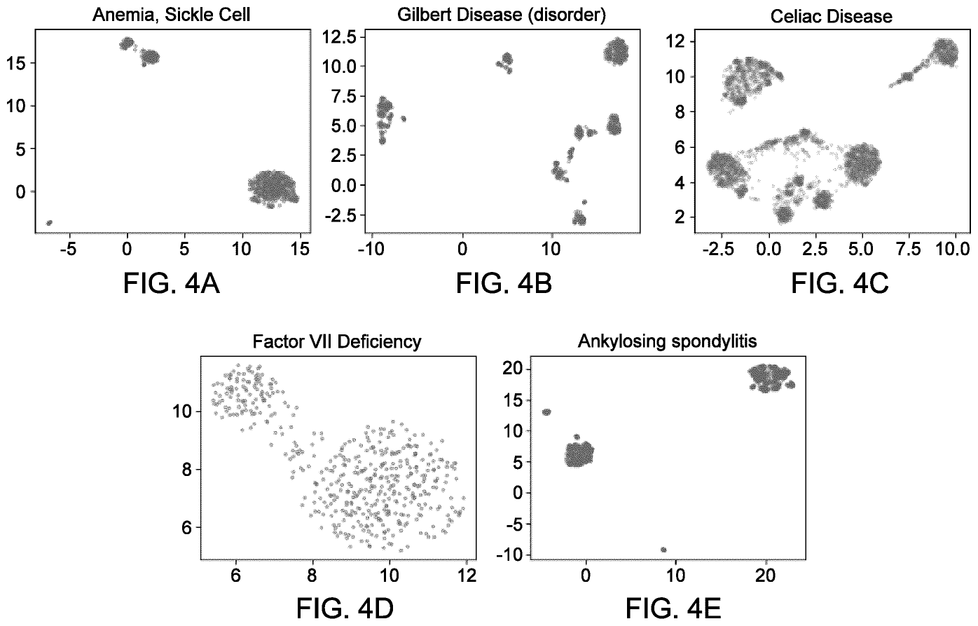

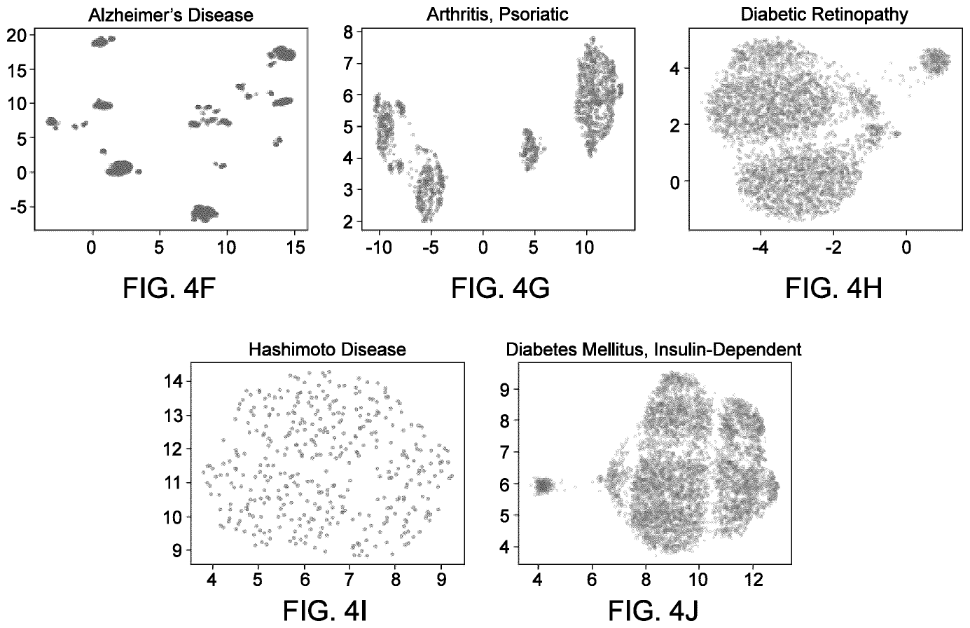

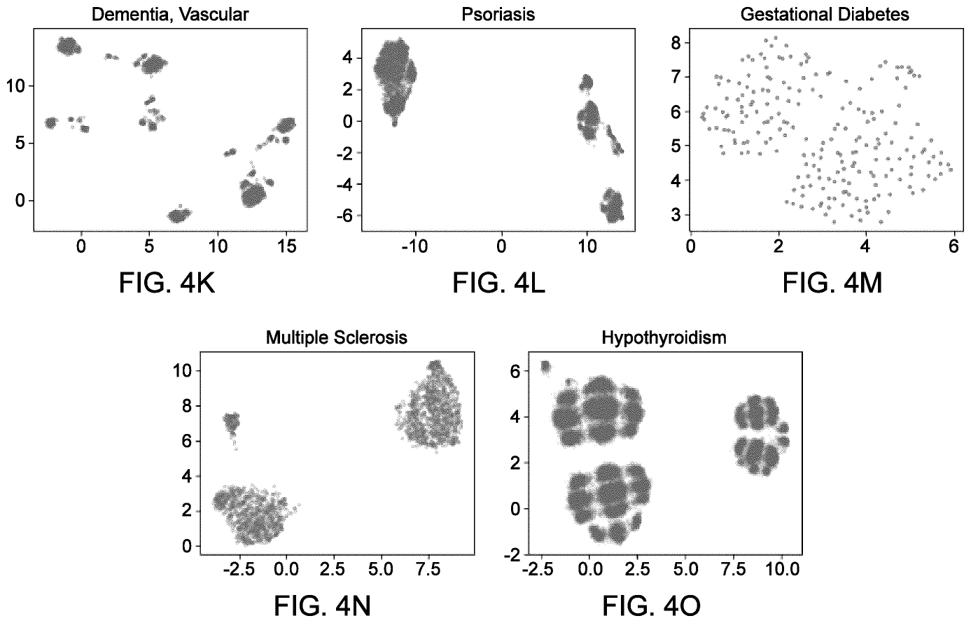

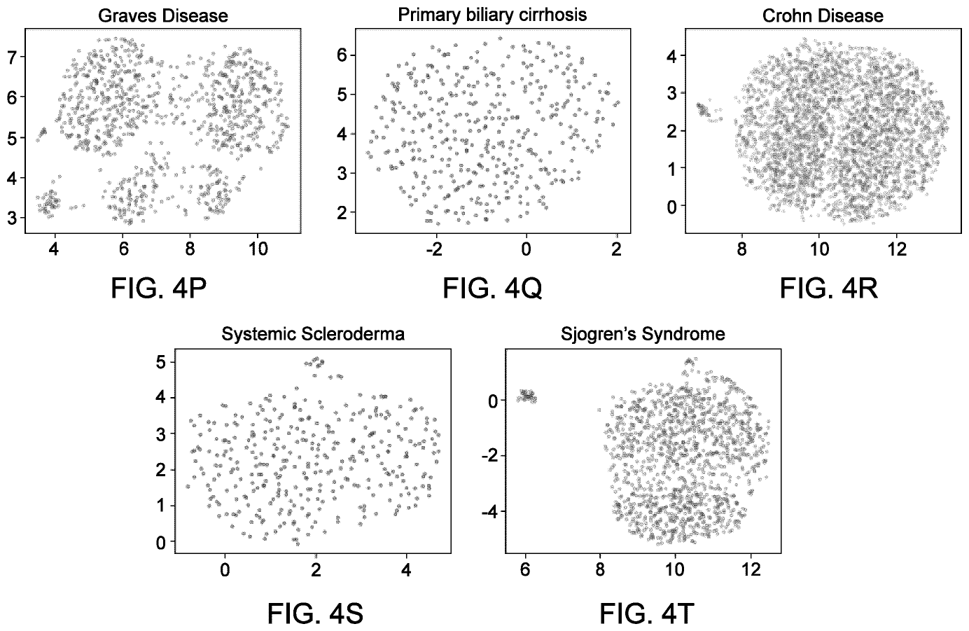

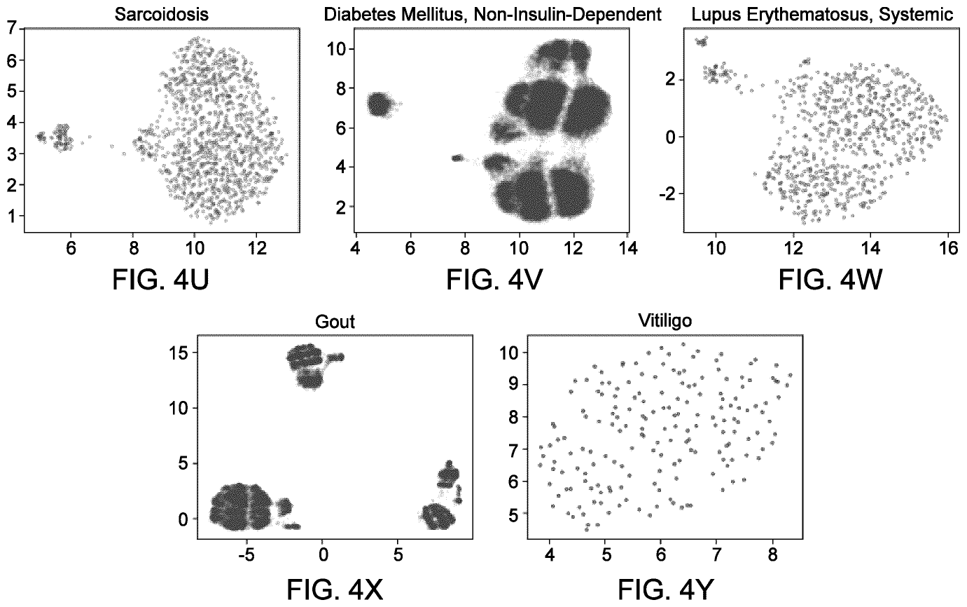

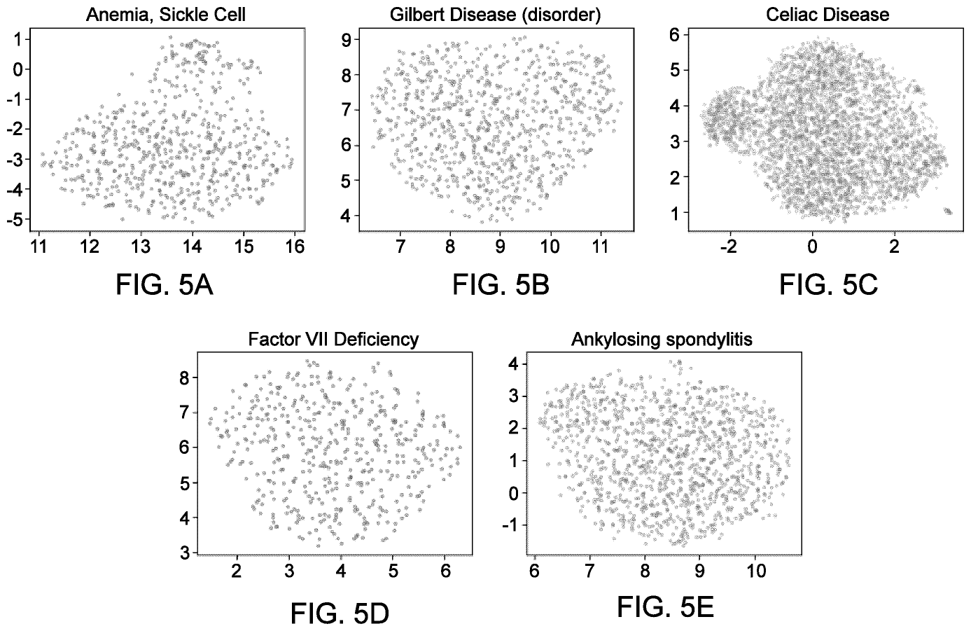

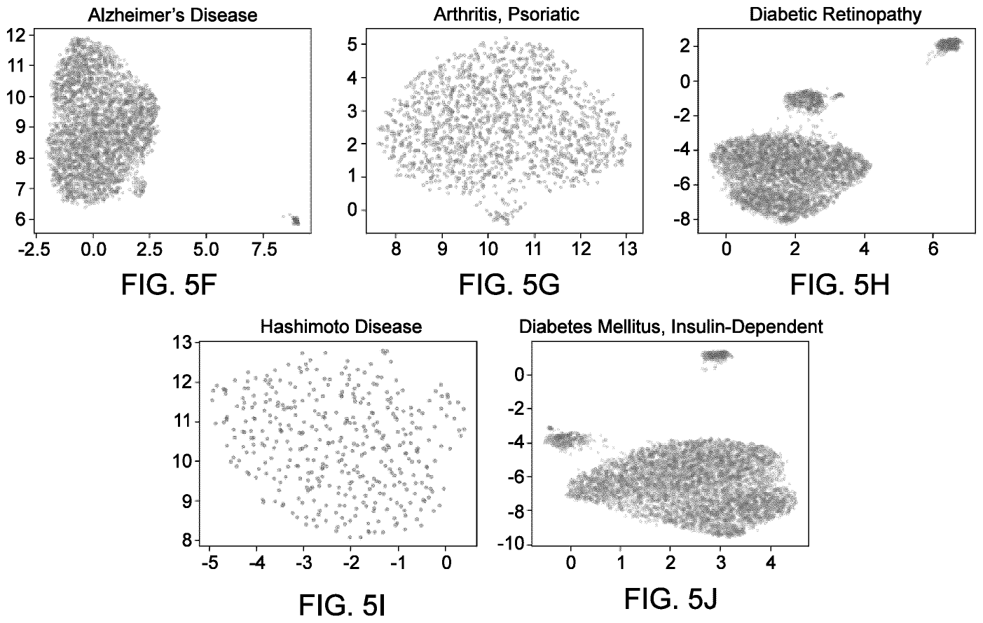

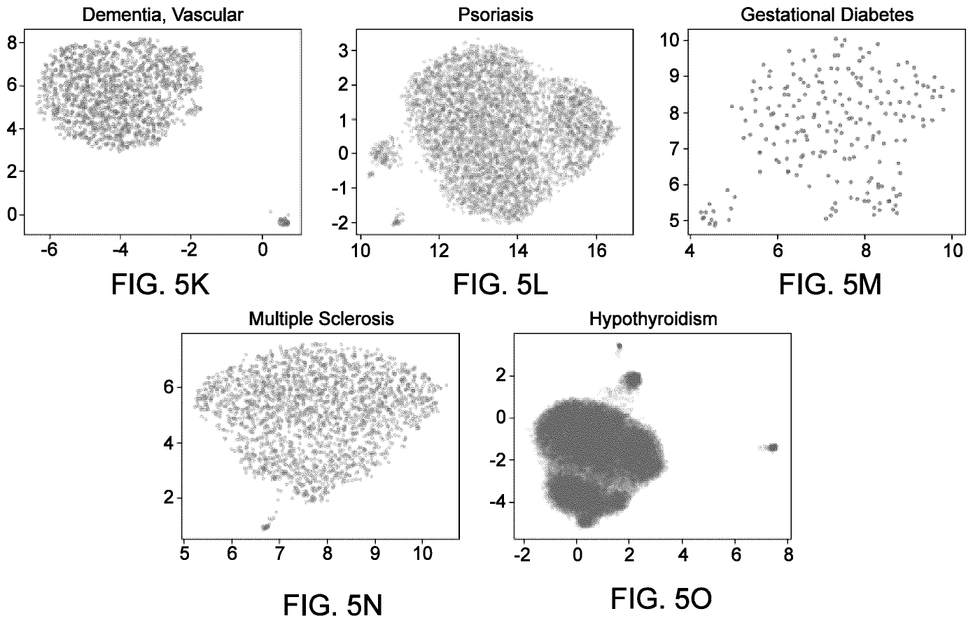

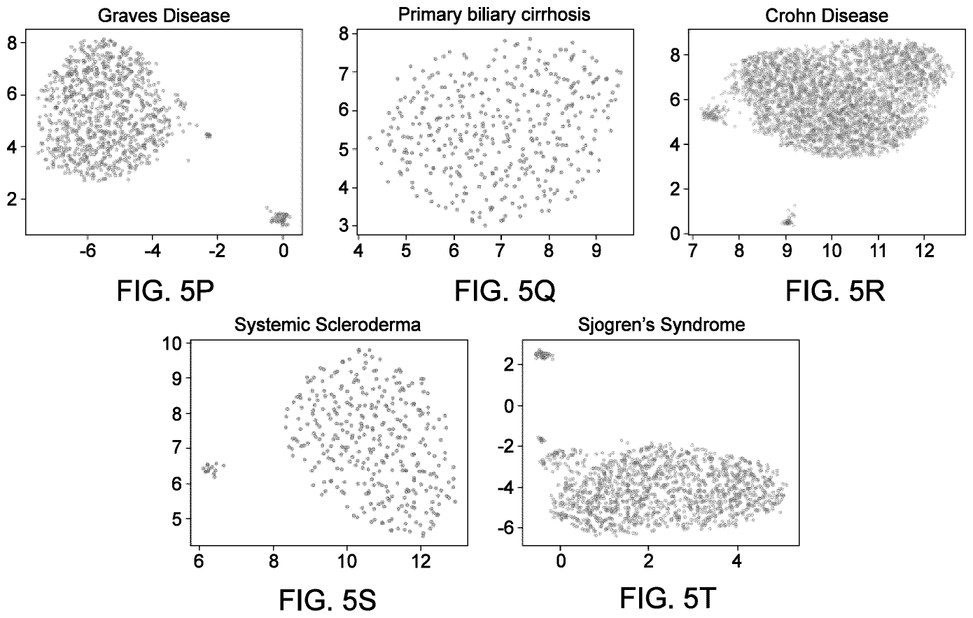

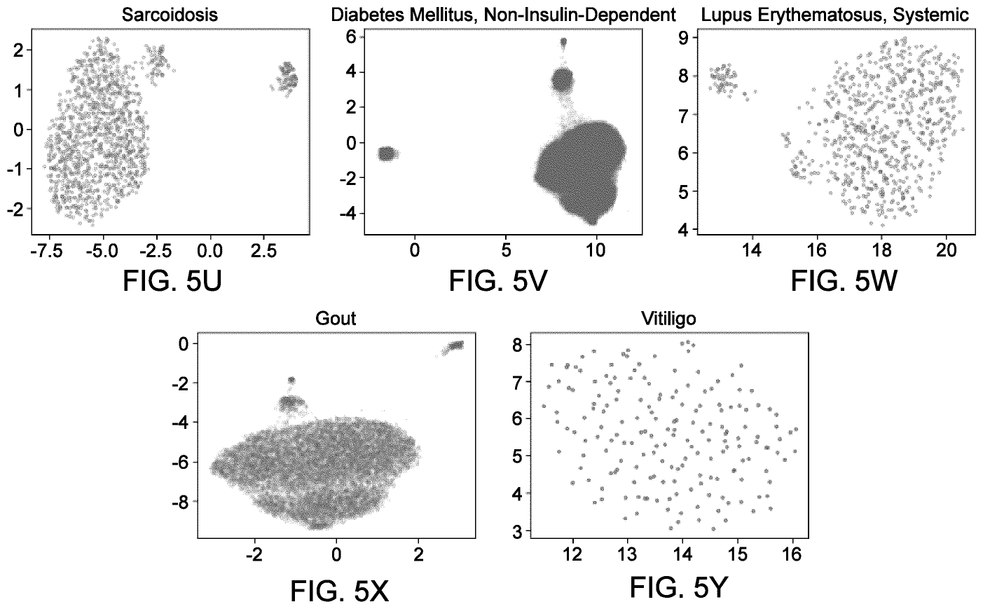

An experiment was performed to demonstrate how the clustering of patients based on feature impact values can be used to identify disease subtypes to advance precision medicine. A disease prediction model (in this example Partial Least Squares Regression) was first learned to predict the likelihood of a given disease using the patient's genetic data. The feature impact values for each individual with disease was then calculated, as described above with respect to step 110. Subsequently, a clustering algorithm (in this example Uniform Manifold Approximation and Projection) was used to group individuals based on their feature impact values, leading to the identification of clusters/subtypes that would not have been obvious without such a method. Each box in FIGS. 4A to 4Y corresponds to a box in FIGS. 5A to 5Y and represents a specific disease, and each point within that box represents an individual with that disease. As can be seen by contrasting the clustering between corresponding FIGS. 4A and 5A, 4B and 5B, . . . , and 4Y and 5Y, there is better patient stratification based on feature impact values (FIGS. 4A to 4Y) compared with clustering using raw feature values (FIG. 5A to 5Y). The disclosed method therefore provides a higher resolution of disease as part of the treatment prioritization system (precision medicine).

In embodiments described above, a system for treatment response prediction and prioritization is provided which includes the creation and use of a “genetic knowledge graph.” The genetic knowledge graph consists of genetic variants, genes, diseases, medications, and is enriched with additional clinical concepts extracted from clinical free-text data, as well as biological data. The system leverages a graph representation learning and specifically uses the graph convolutional network in the model training and prediction process. The graph used by the system is a single global graph (i.e., genetic knowledge graph), built directly into the GCN model, and is shared among all individuals during the system training and prediction process for all prediction instances. The system starts with the creation of the genetic knowledge graph, followed by creating the GCN based on the single genetic knowledge graph. Next, the patient feature values are integrated with the GCN for supervised model training. After the model has been trained, it is used to predict the likelihood of each treatment followed by a ranking to prioritize step to order the treatments.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the invention as defined in the claims appended hereto.

1. A method for treatment prioritization, the method comprising:
generating an enriched genetic knowledge graph representing relationships between genetic variants, genes, diseases, symptoms, and/or treatments, the enriched genetic knowledge graph comprising vertices and edges, with each vertex corresponding to one of a genetic variant, a gene, a disease, a treatment option, and a symptom;
analyzing the enriched genetic knowledge graph to embed the vertices of the enriched genetic knowledge graph in corresponding genetic knowledge vectors, each genetic knowledge vector encoding information about the corresponding vertex, the corresponding vertex's surrounding graph structure, and/or the corresponding vertex's relationship with other vertices;
receiving a plurality of training patient feature value sets characterizing a plurality of patients;
generating a plurality of training patient vectors, each training patient vector being generated by scaling the genetic knowledge vectors by one of the plurality of training patient feature value sets;
generating a training dataset comprising at least a subset of the plurality of training patient vectors, each training patient vector being labelled with a target value comprising an efficacity of at least one treatment option; and
training a treatment prioritization neural network model using the training dataset to process an input patient vector and generate a predicted efficacity of the at least one treatment option.

2. The method of claim 1, further comprising:
generating a genetic knowledge graph representing known relationships between at least genetic variants, genes, diseases, and treatments, the genetic knowledge graph comprising vertices and edges, with each vertex corresponding to one of a genetic variant, a gene, a disease, and a treatment option;
generating a clinical concepts graph representing relationships between at least diseases, symptoms, and treatments identified from clinical patient information, the clinical concepts graph comprising vertices and edges, with each vertex corresponding to one of a disease, a treatment option, and a symptom; and
generating the enriched genetic knowledge graph by integrating the genetic knowledge graph and the clinical concepts graph, wherein vertices of the genetic knowledge graph and vertices of the clinical concepts graph corresponding to one of a common disease and a common treatment option are merged.

3. The method of claim 2, wherein at least a part of the clinical patient information is textual clinical patient information, the method further comprising processing textual clinical patient information by a named-entity recognizer, wherein each of at least a subset of the vertices of the clinical concepts graph is associated with a recognized entity.

4. The method of claim 3, wherein each of at least a subset of the edges of the clinical concepts graph is associated with a cooccurrence of two recognized entities.

5. The method of claim 1, further comprising, for at least one given disease:
providing a disease prediction model configured to predict a likelihood of a patient developing the given disease;
calculating, using the disease prediction model, feature impact values corresponding to a contribution of at least one feature of the genetic knowledge graph to the likelihood of the patient developing the given disease;
clustering the feature impact values to extract subgroups of patients with similar feature impact values; and
for each cluster, adding a disease subtype vertex and an edge connecting to the disease subtype vertex to a vertex corresponding to the given disease to the genetic knowledge graph.

6. The method of claim 1, wherein analyzing the enriched genetic knowledge graph comprises using at least one graph neural network.

7. The method of claim 6, wherein learning the genetic knowledge vectors comprises using one graph neural network for each edge type.

8. The method of claim 1, wherein each patient vector in the training dataset is labelled with a target value further comprising a side effect of the at least one treatment option, wherein the treatment prioritization neural network model is trained to further process the input patient vector to generate a predicted side effect of the at least one treatment option.

9. The method of claim 1, further comprising:
receiving patient feature values characterizing a patient;
generating the patient input vector by scaling the genetic knowledge vectors by the patient feature values; and
providing the patient input vector as an input to the treatment prioritization neural network model, thereby generating a treatment efficacy vector predicting efficacity of a plurality of treatment options.

10. The method of claim 9, further comprising:
computing a Z-score for each of a plurality of treatment options in the treatment efficacy vector; and
ranking the treatment options based on the corresponding Z-score.

11. A system for treatment prioritization, the system comprising:
one or more processor;
memory having a treatment prioritization neural network model stored thereon; and
a training module configured to cause the one or more processor to:
generate an enriched genetic knowledge graph representing relationships between genetic variants, genes, diseases, symptoms, and/or treatments, the enriched genetic knowledge graph comprising vertices and edges, with each vertex corresponding to one of a genetic variant, a gene, a disease, a treatment option, and a symptom;
analyze the enriched genetic knowledge graph to embed the vertices of the enriched genetic knowledge graph in corresponding genetic knowledge vectors, each genetic knowledge vector encoding information about the corresponding vertex, the corresponding vertex's surrounding graph structure, and/or the corresponding vertex's relationship with other vertices;
receive a plurality of training patient feature value sets characterizing a plurality of patients;
generate a plurality of training patient vectors, each training patient vector being generated by scaling the genetic knowledge vectors by one of the plurality of training patient feature value sets;
generate a training dataset comprising at least a subset of the plurality of training patient vectors, each training patient vector being labelled with a target value comprising an efficacity of at least one treatment option; and
train the treatment prioritization neural network model using the training dataset to process an input patient vector and generate a predicted efficacity of the at least one treatment option.

12. The system of claim 11, wherein the training module is further configured to cause the one or more processor to:
generate a genetic knowledge graph representing known relationships between at least genetic variants, genes, diseases and treatments, the genetic knowledge graph comprising vertices and edges, with each vertex corresponding to one of a genetic variant, a gene, a disease, and a treatment option;
generate a clinical concepts graph representing relationships between at least diseases, symptoms, and treatments identified from clinical patient information, the clinical concepts graph comprising vertices and edges, with each vertex corresponding to one of a disease, a treatment option, and a symptom; and
generate the enriched genetic knowledge graph by integrating the genetic knowledge graph and the clinical concepts graph, wherein vertices of the genetic knowledge graph and vertices of the clinical concepts graph corresponding to one of a common disease and a common treatment option are merged.

13. The system of claim 12, wherein at least a part of the clinical patient information is textual clinical patient information, the system further comprising a named-entity recognizer configured to process textual clinical patient information, wherein each of at least a subset of the vertices of the clinical concepts graph is associated with a recognized entity.

14. The system of claim 13, wherein each of at least a subset of the edges of the clinical concepts graph is associated with a cooccurrence of two recognized entities.

15. The system of claim 11, the memory further having a disease prediction model configured to predict a likelihood of a patient developing a given disease stored thereon, wherein the training module is further configured to cause the one or more processor, for at least one given disease, to:
calculate, using the disease prediction model, feature impact values corresponding to a contribution of at least one feature of the genetic knowledge graph to the likelihood of the patient developing the given disease;
cluster the feature impact values to extract subgroups of patients with similar feature impact values; and
for each cluster, add a disease subtype vertex and an edge connecting to the disease subtype vertex to a vertex corresponding to the given disease to the genetic knowledge graph.

16. The system of claim 11, further comprising at least one graph neural network configured to analyze the enriched genetic knowledge graph.

17. The system of claim 16, wherein the at least one graph neural network corresponds to a plurality of graph neural networks, wherein learning the genetic knowledge vectors comprises using one graph neural network for each edge type.

18. The system of claim 11, wherein each patient vector in the training dataset is labelled with a target value further comprising a side effect of the at least one treatment option, wherein the treatment prioritization neural network model is trained to further process the input patient vector to generate a predicted side effect of the at least one treatment option.

19. A system for treatment prioritization, the system comprising:
one or more processor;
memory having a trained treatment prioritization neural network model stored thereon;
an input device configured to receive patient feature values characterizing a patient;
an inference module configured to cause the one or more processor to:
generate a patient input vector by scaling genetic knowledge vectors by the patient feature values, and
provide the patient input vector as an input to the treatment prioritization neural network model, thereby generating a treatment efficacy vector predicting efficacity of a plurality of treatment options; and
an output device configured to convey the predicted efficacity of the plurality of treatment options from a user.

20. The system of claim 19, wherein the inference module is further configured to cause the one or more processor to:
compute a Z-score for each of a plurality of treatment options in the treatment efficacy vector; and
rank the treatment options based on the corresponding Z-score; and
wherein the output device is configured to convey the plurality of treatment options according to their rank.
